(19) United States
(12) Patent Application Publication
(10) Pub. No.: US 2020/0009042 A1
(43) Pub. Date: Jan. 9, 2020

(54) METHOD OF SKIN REJUVENATION USING STEM CELL ENGRAFTMENT

(71) Applicant: Robert Mehran Dean, Palm Harbor, FL (US)

(72) Inventor: Robert Mehran Dean, Palm Harbor, FL (US)

(21) Appl. No.: 16/262,506

(22) Filed: Jan. 30, 2019

Related U.S. Application Data
(60) Provisional application No. 62/694,041, filed on Jul. 5, 2018.

(51) Int. Cl. A61K 8/98; A61K 8/73; A61K 8/02; A61Q 19/08

(52) U.S. Cl. CPC A61K 8/981; A61Q 19/08; A61K 8/0204; A61K 8/735

(57) ABSTRACT

A system and method of improving the condition of skin by reducing or eliminating wrinkles and hyperpigmentation is presented. The method delivers stem cells into the dermal layer of the skin to increase the number and density of fibroblasts in the dermal layer which in turn increases collagen, elastin and hyaluronic acid production to improve the appearance of the skin. The method includes constructing a scaffolding within microperforations in the skin and administering a therapeutically effective amount of stem cells to the skin which are supported on the scaffolding to remain in the microperforations for weeks thus allowing the dermal layer of skin to rebuild to a more youthful state.

### CROSS REFERENCE TO RELATED APPLICATIONS

This application is a nonprovisional of and claims priority to U.S. Provisional Application No. 62/694,041, entitled “Procedure of Skin Rejuvenation Using Stem Cells Engraftment”, filed Jul. 5, 2018, the contents of which are herein incorporated by reference.

### FIELD OF INVENTION

This invention relates to a system and method of improving skin appearance and reducing skin aging. Specifically, a system and method of reducing wrinkles and hyperpigmentation is presented.

### BACKGROUND OF THE INVENTION

Facial aging occurs as the result of several factors including, but not limited to, inherent changes within the skin, effects of gravity, facial muscles acting on the skin (dynamic lines), soft tissue loss or shift, tissue elasticity and bone loss. Skin appears to age as the epidermis begins to thin as a result of an inherent loss of density in the dermal and subdermal layers.

Fibroblasts are typically spindle-shaped cells having an oval flat nucleus and are present in connective tissue. Fibroblasts are capable of producing the extracellular matrix (ECM) including collagen, glycosaminoglycans, proteoglycans, elastin and hyaluronic acid, as well as providing a supporting framework for the ECM and functioning in repair mechanisms. Fibroblasts adhere to, contract, and remodel the ECM to provide mechanical strength to tissue by providing a supporting framework for the ECM and are capable of secreting growth factors, cytokines, chemokines, prostaglandins and proteolytic enzymes such as metalloproteinases to promote tissue rearrangement and repair.

Fibroblast precursors termed “mesenchymal stromal cells” or “mesenchymal stem cells” (MSCs) are adult, fibroblast-like multipotent cells that are capable of differentiating into different tissues of mesodermal origin. MSCs are also capable of exhibiting stem cell function in vivo. MSCs have also been shown to adopt a different phenotype in response to sensing an inflammatory environment and possess both anti-inflammatory and proinflammatory effects. (Bernardo, M. E. and Fibbe, W. E. Mesenchymal Stromal Cells: Sensors and Switchers of Inflammation, Cell Stem Cell, 2013, 13(4):392-402) MSCs can expand rapidly in vitro while maintaining their multipotency. MSCs are characterized by the expression of surface markers such as CD73, CD90 and CD105 as well as the absence of the expression of hematopoietic lineage markers. (Yagi, H. et al., Mesenchymal stem cells: mechanisms of immunomodulation and homing, Cell Transplantation, 2010, 19:667-679)

MSCs can be derived from various sources including, but not limited to, bone marrow (BM), umbilical cord (UC), peripheral blood, adipose tissue, and umbilical cord blood (UCB). Umbilical cord blood is considered one of the most abundant sources of non-embryonic stem cells. Human umbilical cord blood□derived MSCs (hUCB□MSCs) have distinct advantages, including accessibility, a higher proliferation capacity, and a lower immunogenicity as compared to bone marrow derived MSCs. hUCB□MSCs are capable of self-renewal, possess multipotent differentiation properties, and possess immunoregulatory traits that permit allogeneic transplantation.

As a person ages, typically between the ages of 30 and 45, they lose approximately half of the collagen, elastin, and hyaluronic acid in the dermis. These proteins are responsible for the skin's turgor, the loss of which causes the skin to become lax, thus losing strength and elasticity. When the skin loses elasticity, it is less able to recover from stretching and creasing. Coupled with gravity, muscle activity, and tissue changes, the skin begins to wrinkle, sag and exhibit hyperpigmentation. Decreased hydration and breakdown of bonds between cells also reduces the barrier function of the skin, which can cause pore size to increase. As a person ages, the loss of fibroblast density results in the face losing volume, soft tissue, and fat. The appearance of jowls and folds are usually caused by the drooping of facial tissues and the folding of areas where the muscles below are attached to the skin. As part of the reduction in soft tissue, the face appears hollowed.

The beauty industry offers numerous procedures designed to renew the appearance of the face as there is a strong desire in today's society to obtain/maintain a youthful appearance. Aesthetic medicine has been primarily based on the surgical approach for many years, however there is an increasing interest in non-surgical procedures that can provide skin rejuvenation in the dermal layers. Surgical procedures, such as face lifts, typically use general anesthesia, are more traumatic and have a longer recovery period than non-surgical approaches. Facial cosmetic surgery, together with the help of modern technology, is currently the most effective way to restore the appearance of elasticity and youthfulness of the skin, face and neck.

Non-surgical techniques are frequently referred to as non-invasive and in some cases, minimally-invasive procedures or treatments. These treatments do not require surgical incisions or general anesthesia. Non-surgical techniques are effective in achieving results for patients who seek to soften the signs of aging in a subtler way as compared to plastic surgery. These minimally invasive alternatives usually result in less pain, less scarring, and a quicker recovery for the patient, as well as reduced health care costs. Examples of non-surgical techniques include, but are not limited to, injectables used to treat deeper lines, furrows and folds and topical treatments such as chemical peels, dermabrasion and laser resurfacing used for more superficial lines and pigmentation issues. Injectables include products such as Botox® or fillers, such as Restylane® and Juvéderm®. Botox® treatments inject the botulinum toxin to temporarily paralyze the muscle, particularly facial muscles. In contrast, Restylane® and Juvéderm® are injectable fillers made of cross-linked hyaluronic acid (HA). Since the cells are not producing more HA, the effect of injecting HA is limited by the rate of degradation of the amount injected which makes these products rather short-lived.

A major problem with current non-surgical techniques for skin rejuvenation is that repeated treatments are needed in order to maintain results. Some treatments only last a few weeks while others last months. For example, Botox® treatments last between 3 to 6 months while Restylane® and Juvéderm®, last between 4-9 months depending on the type used. Chemical peels generally last a about 2 months, dermabrasion generally lasts about 6-12 months and laser resurfacing generally lasts 1-2 years.

Given the shortcomings of the current therapies for skin rejuvenation, what is needed is a system and method of skin rejuvenation which has long lasting results and can treat both mimetic and superficial wrinkles as well as hyperpigmentation but is still non-invasive.

### SUMMARY OF INVENTION

The inventors have developed a novel non-surgical method and system for skin rejuvenation using umbilical cord blood derived stem cells such as umbilical cord blood derived mesenchymal stem cells or stromal cells (hUCB□MSCs) to change the microenvironment in the dermis. The stem cells can be delivered into the skin with minimal cellular trauma and act as a catalyst to create the regenerative microenvironment for the regeneration of the extracellular matrix (ECM). Once placed in the dermis, the stem cells are able to engraft or bind to a blood supply and produce the regenerative factors that change the stromal tissue microenvironment.

Administering hUCB-MSCs directly to the dermis allows for the hUCB-MSCs to engraft in the dermis thus enabling fibroblast proliferation leading to increased fibroblast density in the dermis. The greater density and number of fibroblasts results in an increase in the production of collagen, elastin, and hyaluronic acid in the dermis which in turn reduces the appearance and depth of wrinkles, restores the tone and elasticity of the skin, reduces hyperpigmentation, and slows down the future decrease in elasticity of the skin due to aging.

In light of the stem cells engrafting and binding to the dermal tissue, they can live for weeks rather than just a few hours. The stem cells are living cells producing growth factors over several weeks. Current therapies using growth factors are not implanting living cells but rather only administering growth factors themselves which have a half-life of only 6-12 hours, thus making the results of the current therapies more short-lived.

It takes years for the regenerated collagen/elastin/hyaluronic acid matrix to once again deplete thus offering a long-lasting non-surgical option for patients. Because the stem cells change the micro-environment of the dermal layer, thus causing proliferation of fibroblasts, the method differs from other non-surgical methods due to its effectiveness and long-lasting results.

In an embodiment, a method of skin rejuvenation in a patient is presented comprising: applying isotonic sterile saline and a hyaluronic acid composition to a treatment area of skin of the patient to form a scaffolding; puncturing the skin of the treatment area to form a plurality of microperforations between about 0.5 mm to about 2.5 mm in depth; administering a therapeutically effective amount of stem cells to the treatment area of the patient wherein the scaffolding supports the stem cells within the microperforations; and applying a covering on the treatment area for a predetermined amount of time. The covering acts to hold the stem cells within the microperforations to allow the stem cells to engraft in a dermal layer of the skin of the patient.

A topical anesthetic may be applied to the treatment area prior to forming the scaffolding. The scaffolding may be formed from at least two separate applications of the isotonic sterile saline and the hyaluronic acid composition to the treatment area.

The stem cells may be umbilical cord blood derived mesenchymal stem cells (hUCB-MSCs) that are administered topically, with at least 1×10⁶ stem cells being administered.

The covering may be a silicone sheeting mask. A dressing may be applied over the covering to hold the covering in position on the treatment area with the covering and dressing remaining on the treatment area for at least 6 hours.

In an embodiment, a method of reducing or eliminating wrinkles in skin of a patient is presented comprising: applying isotonic sterile saline and a hyaluronic acid composition to a treatment area of skin of the patient to form a scaffolding; microneedling the skin of the treatment area of the patient to form a plurality of microperforations in the skin of the treatment area of the patient; administering a therapeutically effective amount of umbilical cord blood derived stem cells to the treatment area of the patient wherein the scaffolding supports the stem cells within the microperforations; and applying a covering, such as a silicone sheeting mask, on the treatment area for a predetermined amount of time, such as for at least 6 hours. The covering is used to hold the stem cells within the microperforations to allow the stem cells to engraft in a dermal layer of the skin of the patient.

The scaffolding may be formed from at least two layers of isotonic sterile saline and a hyaluronic acid composition and at least 1×10⁶ stem cells may be administered.

In an embodiment, a method of reducing or eliminating hyperpigmentation in skin of a patient is presented comprising: applying isotonic sterile saline and a hyaluronic acid composition to a treatment area of skin of the patient to form a scaffolding; microneedling the skin of the treatment area of the patient to form a plurality of microperforations in the skin of the treatment area of the patient; administering a therapeutically effective amount of umbilical cord blood derived stem cells to the treatment area of the patient wherein the scaffolding supports the stem cells within the microperforations; and applying a covering, such as a silicone sheeting mask, on the treatment area for a predetermined amount of time, such as for at least 6 hours. The covering is used to hold the stem cells within the microperforations to allow the stem cells to engraft in a dermal layer of the skin of the patient.

The scaffolding may be formed from at least two layers of isotonic sterile saline and a hyaluronic acid composition and at least 1×10⁶ stem cells may be administered.

In an embodiment, a method of increasing fibroblast density in the dermis is presented comprising: applying isotonic sterile saline and a hyaluronic acid composition to a treatment area of skin of the patient to form a scaffolding; microneedling the skin of the treatment area of the patient to form a plurality of microperforations in the skin of the treatment area of the patient; administering a therapeutically effective amount of umbilical cord blood derived stem cells to the treatment area of the patient wherein the scaffolding supports the stem cells within the microperforations; and applying a covering, such as a silicone sheeting mask, on the treatment area for a predetermined amount of time, such as for at least 6 hours. The covering is used to hold the stem cells within the microperforations to allow the stem cells to engraft in a dermal layer of the skin of the patient.

In an embodiment, a method of increasing collagen production in the dermis is presented comprising: applying isotonic sterile saline and a hyaluronic acid composition to a treatment area of skin of the patient to form a scaffolding; microneedling the skin of the treatment area of the patient to form a plurality of microperforations in the skin of the treatment area of the patient; administering a therapeutically effective amount of umbilical cord blood derived stem cells to the treatment area of the patient wherein the scaffolding supports the stem cells within the microperforations; and applying a covering, such as a silicone sheeting mask, on the treatment area for a predetermined amount of time, such as for at least 6 hours. The covering is used to hold the stem cells within the microperforations to allow the stem cells to engraft in a dermal layer of the skin of the patient.

In an embodiment, a method of regenerating the extracellular matrix (ECM) in the dermis is presented comprising: applying isotonic sterile saline and a hyaluronic acid composition to a treatment area of skin of the patient to form a scaffolding; microneedling the skin of the treatment area of the patient to form a plurality of microperforations in the skin of the treatment area of the patient; administering a therapeutically effective amount of umbilical cord blood derived stem cells to the treatment area of the patient wherein the scaffolding supports the stem cells within the microperforations; and applying a covering, such as a silicone sheeting mask, on the treatment area for a predetermined amount of time, such as for at least 6 hours. The covering is used to hold the stem cells within the microperforations to allow the stem cells to engraft in a dermal layer of the skin of the patient.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. For a fuller understanding of the invention, reference should be made to the following detailed description, taken in connection with the accompanying drawings, in which:

FIG. 1 is an image depicting a cross section of younger and older skin exemplifying the changes in the dermis over time.

FIG. 2 is an image of the mesenchymal stem cell secretome. Reproduced from Maumas, M. et al., Mesenchymal stem cells in regenerative medicine applied to rheumatic diseases: Role of secretome and exosomes, Biochemie, 2013, 95:2229-2234.

FIG. 3 is an image depicting exemplary cell types that can be differentiated from mesenchymal stem cells. Reproduced from Tyndall, A., Mesenchymal stem cell treatments in rheumatology—a glass half full?, Nat. Rev. Rheumatol., February 2014, 10(2):117-24.

FIG. 4A-B depict a measurement scale for facial wrinkles. (A) a table of the Lemperle Classification of Facial Wrinkles and (B) anatomic reference points for assessment and measurement of wrinkle depth. If the deepest point of the wrinkle is outside of this point, it can be marked or described separately. HF, horizontal forehead lines; GF, glabellar frown lines; PO, periorbital lines; PA, preauricular lines; CL, cheek lines; NL, nasolabial folds; UL, upper radial lip lines; LL, lower radial lip lines: CM, corner of mouth lines; ML, marionette lines; LM, labiomental crease; NF, horizontal neck folds. Reproduced from Lemperle, G. et al., A classification of facial wrinkles, Plastic and Reconstructive Surgery, November 2001, 108(6):1735-1750.

FIG. 5A-B are images depicting wrinkle depth of Patient A before and 7 weeks post-treatment. (A) unretouched photographs taken before treatment (time 0) and 7 weeks post-treatment; (B) graph depicting the change in wrinkle depth at 7 weeks post-treatment based on the Lemperle Classification of Facial Wrinkles.

FIG. 6A-B are images depicting wrinkle depth of Patient B before and 7 weeks post-treatment. (A) unretouched photographs taken before treatment (time 0) and 7 weeks post-treatment; (B) graph depicting the change in wrinkle depth at 7 weeks post-treatment based on the Lemperle Classification of Facial Wrinkles.

FIG. 7A-B are images depicting wrinkle depth of Patient C before and 7 weeks post-treatment. (A) unretouched photographs taken before treatment (time 0) and 7 weeks post-treatment; (B) graph depicting the change in wrinkle depth at 7 weeks post-treatment based on the Lemperle Classification of Facial Wrinkles.

FIG. 8A-B are images depicting wrinkle depth of Patient D before and 7 weeks post-treatment. (A) unretouched photographs taken before treatment (time 0) and 7 weeks post-treatment; (B) graph depicting the change in wrinkle depth at 7 weeks post-treatment based on the Lemperle Classification of Facial Wrinkles.

FIG. 9A-B are images depicting wrinkle depth of Patient E before and 7 weeks post-treatment. (A) unretouched photographs taken before treatment (time 0) and 7 weeks post-treatment; (B) graph depicting the change in wrinkle depth at 7 weeks post-treatment based on the Lemperle Classification of Facial Wrinkles.

FIG. 10A-B are images depicting wrinkle depth of Patient F before and 7 weeks post-treatment. (A) unretouched photographs taken before treatment (time 0) and 7 weeks post-treatment; (B) graph depicting the change in wrinkle depth at 7 weeks post-treatment based on the Lemperle Classification of Facial Wrinkles.

FIG. 11A-B are images depicting wrinkle depth of Patient G before and 7 weeks post-treatment. (A) unretouched photographs taken before treatment (time 0) and 7 weeks post-treatment; (B) graph depicting the change in wrinkle depth at 7 weeks post-treatment based on the Lemperle Classification of Facial Wrinkles.

FIG. 12A-B are images depicting wrinkle depth of Patient H before and 7 weeks post-treatment. (A) unretouched photographs taken before treatment (time 0) and 7 weeks post-treatment; (B) graph depicting the change in wrinkle depth at 7 weeks post-treatment based on the Lemperle Classification of Facial Wrinkles.

FIG. 13A is an image depicting wrinkle assessment for Patients A-H. (A) graph depicting the Lemperle score of Patients A-H before and 7 weeks post-treatment;

FIG. 13B-C are a series of graphs depicting wrinkle assessment for Patients A-H. (B) graph depicting the numerical change in the Lemperle score of Patients A-H; (C) graph depicting the percentage of improvement in Patients A-H 7 weeks post-treatment.

FIG. 13D is an image depicting wrinkle assessment for Patients A-H. (D) Table summarizing 13A-C.

FIG. 14 is a one skin hue card of a color table in the Taylor Hyperpigmentation Scale. The scale consists of 15 uniquely colored skin hue cards spanning the full range of skin hues, each with 10 bands of increasingly darker graduations of skin hues that represent progressive levels of hyperpigmentation. Reproduced from Taylor, S. C. et al., The Taylor Hyperpigmentation Scale: a new visual assessment tool for the evaluation of skin color and pigmentation, Cutis, 2005, 76:270-274.

FIG. 15A-B are images depicting hyperpigmentation of Patient A before and 7 weeks post-treatment. (A) unretouched photographs taken before treatment (time 0) and 7 weeks post-treatment; (B) graph depicting the change in hyperpigmentation at 7 weeks post-treatment based on the Taylor Hyperpigmentation Scale.

FIG. 16A-B are images depicting hyperpigmentation of Patient B before and 7 weeks post-treatment. (A) unretouched photographs taken before treatment (time 0) and 7 weeks post-treatment; (B) graph depicting the change in hyperpigmentation at 7 weeks post-treatment based on the Taylor Hyperpigmentation Scale.

FIG. 17A-B are images depicting hyperpigmentation of Patient C before and 7 weeks post-treatment. (A) unretouched photographs taken before treatment (time 0) and 7 weeks post-treatment; (B) graph depicting the change in hyperpigmentation at 7 weeks post-treatment based on the Taylor Hyperpigmentation Scale.

FIG. 18A-B are images depicting hyperpigmentation of Patient D before and 7 weeks post-treatment. (A) unretouched photographs taken before treatment (time 0) and 7 weeks post-treatment; (B) graph depicting the change in hyperpigmentation at 7 weeks post-treatment based on the Taylor Hyperpigmentation Scale.

FIG. 19A is an image depicting hyperpigmentation assessment for Patients A-D. (A) graph depicting the Taylor Hyperpigmentation score of Patients A-D before and 7 weeks post-treatment.

FIG. 19B-C are a series of graphs depicting hyperpigmentation assessment for Patients A-D. (B) graph depicting the numerical change in the Taylor Hyperpigmentation score of Patients A-D; (C) graph depicting the percentage of improvement in Patients A-D 7 weeks post-treatment.

FIG. 19D is an image depicting hyperpigmentation assessment for Patients A-D. (D) Table summarizing 19A-C.

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized, and structural changes may be made without departing from the scope of the invention.

### Definitions

Unless otherwise defined, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are described herein. All publications mentioned herein are incorporated herein by reference in their entirety to disclose and describe the methods and/or materials in connection with which the publications are cited. It is understood that the present disclosure supercedes any disclosure of an incorporated publication to the extent there is a contradiction.

All numerical designations, such as pH, temperature, time, concentration, and molecular weight, including ranges, are approximations which are varied up or down by increments of 1.0 or 0.1, as appropriate. It is to be understood, even if it is not always explicitly stated that all numerical designations are preceded by the term “about”. It is also to be understood, even if it is not always explicitly stated, that the reagents described herein are merely exemplary and that equivalents of such are known in the art and can be substituted for the reagents explicitly stated herein.

The term “about” or “approximately” as used herein refers to being within an acceptable error range for the particular value as determined by one of ordinary skill in the art, which will depend in part on how the value is measured or determined, i.e. the limitations of the measurement system, i.e. the degree of precision required for a particular purpose, such as a pharmaceutical formulation. As used herein, “about” refers to +10%.

As used in the specification and claims, the singular form “a”, “an” and “the” include plural references unless the context clearly dictates otherwise. For example, the term “a nanoparticle” includes a plurality of nanoparticles, including mixtures thereof.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

“Patient” is used to describe an animal, preferably a human, to whom treatment is administered, including prophylactic treatment with the compositions of the present invention. “Patient” and “subject” are used interchangeably herein.

“Administration” or “administering” is used to describe the process in which the formulations of the present invention are delivered to a patient, including the various creams, compositions and stem cells. The formulations may be administered topically, such as a cream, liquid, gel, lotion, serum, foam, paste, or an ointment. Alternatively, the stem cells in particular may be injected.

The “therapeutically effective amount” for purposes herein is thus determined by such considerations as are known in the art. A therapeutically effective amount of the formulations described herein is that amount necessary to provide a therapeutically effective result in vivo. The amount of stem cells must be effective to achieve a beneficial biological response, including but not limited to, symptoms of skin aging such as improvement of epidermal elasticity; improvement of epidermal smoothness; reduction or elimination of epidermal discoloration/hyperpigmentation; increase in fibroblast proliferation; increase in fibroblast density; increase in collagen production; regeneration of the extracellular matrix (ECM); and reduction or elimination of fine lines, wrinkles, folds, and furrows. In accordance with the present invention, a suitable single dose size is a dose that is capable of reducing or eliminating a symptom in a patient when administered one or more times over a suitable time period. One of skill in the art can readily determine appropriate single dose sizes for topical administration based on the size of the treatment area of the patient and the route of administration. In an embodiment, the therapeutically effective amount of stem cells is at least 1×10⁶ viable cells administered to the treatment area.

The pharmaceutical compositions of the subject invention can be formulated according to known methods for preparing pharmaceutically useful compositions. Furthermore, as used herein, the phrase “pharmaceutically acceptable carrier” means any of the standard pharmaceutically acceptable carriers. The pharmaceutically acceptable carrier can include diluents, adjuvants, and vehicles, as well as implant carriers, and inert, non-toxic solid or liquid fillers, diluents, or encapsulating material that does not react with the active ingredients of the invention. In some embodiments, the pharmaceutically acceptable carrier is a gel, ointment, hydrogel, cream, aerosol, lotion, foam, fluid, serum, or powder. Examples of other pharmaceutically acceptable carriers include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pa., Mack Publishing Company, 19^{th} ed.) describes formulations which can be used in connection with the subject invention.

“Treatment” or “treating” as used herein refers to any of: the alleviation, amelioration, elimination and/or reduction of at least one symptom after the onset of a particular disorder. An effective or successful treatment provides a clinically observable improvement. For example, “treatment” of skin aging may include any one or more of the following: amelioration and/or elimination of one or more symptoms associated with skin aging, reduction of one or more symptoms of aging, and alleviation of symptoms of skin aging.

The term “gentle cleanser” as used herein refers to a cleansing agent that is pH balanced, mild and non-irritating to the skin without drying the skin or stripping oils from the skin. Preferably the cleanser is acid-free, fragrance free and soap-free.

The term “topical anesthetic” as used herein refers to an anesthetic applied locally topically to an area such as the face to numb the area. Examples of topical anesthetics include, but are not limited to, creams, serums, lotions, liquids, ointments, and gels containing an anesthetic including, but not limited to, lidocaine, xylocaine, prilocaine, tetracaine, benzocaine or combinations thereof.

The term “microneedling” as used herein refers to a procedure in which numerous very fine needles are used to cause numerous microperforations (over 100 per square inch) in the skin. In some embodiments, a microneedling device such as a dermaroller is used to microneedle the skin to cause microperforations between about 0.5 mm to about 2.5 mm. Each treatment section is covered with at least 4 passes, but no more than 6 passes, of the microneedling device. Specific areas of concern can be spot-treated with additional passes at deeper needle depths as tolerated.

The term “stem cells” as used herein refers to a pluripotent, or lineage-uncommitted, progenitor cell, which is potentially capable of an unlimited number of mitotic divisions to either renew itself or to produce progeny cells which will differentiate into the desired cell type. In contrast to pluripotent stem cells, lineage-committed progenitor cells are generally considered to be incapable of giving rise to numerous cell types that phenotypically differ from each other. Instead, progenitor cells give rise to one or possibly two lineage-committed cell types. Both adult and embryonic stem cells are contemplated for use herein.

As used herein, the term “multipotential” or “multipotentiality” is meant to refer to the capability of a stem cell to differentiate into more than one type of cell. “Pluripotent stem cell” has the ability to differentiate into at least two cell types belong to different germ layer lineages (mesoderm, endoderm, and ectoderm) from which all the cells of the body arise.

Umbilical cord blood derived stem cells (UCBCs) are preferably used herein, however other types of stem cells are contemplated for use including, but not limited to, bone marrow derived stem cells and adipose tissue derived stem cells. Umbilical cord blood derived stem cells include collection of mesenchymal stem cells (MSCs) from UCB. Use of UCBCs is an easier, less expensive and non-invasive method than collecting MSCs from bone marrow or adipose tissue.

“Umbilical cord blood derived stem cells” refers to hematopoietic stem cells isolated from umbilical cord blood. “Umbilical cord blood” or “cord blood” refers to blood obtained from a neonate or fetus, and specifically refers to any blood obtained from an umbilical cord or placenta of a newborn. The use of cord or placental blood for autologous or allogenic transplantation is conceived. The blood may be collected by drainage from the cord and/or placenta or by aspiration.

“Bone marrow stem cell” or “BMSC” is used to refer to adult stem cells, also called somatic stem cells, isolated from the hematopoietic compartment of an organism. Specifically, the term refers to adult stem cells isolated from the bone marrow of an organism that is not a neonate or fetus.

“Adipose derived stem cells” or “hADSCs” is used to refer to adult stem cells derived from adipose tissue of a human that is not a neonate or fetus. Human adipose derived stem cells (hADSCs) have the potential to proliferate, differentiate into different cell lineages, and excrete an extensive secretome containing growth factors, cytokines, chemokines, microRNAs and long noncoding RNA (lncRNA). Adipose-derived stem cells are a subset population of cells derived from adipose tissue, either white or brown, which can be separated from other components of the adipose tissue using standard culturing procedures.

The term “mesenchymal stem cells” or “mesenchymal stromal cells” (MSCs) as used herein refers to pluripotent fibroblast-like cells capable of secreting trophic factors. MSCs can be derived from various different tissues including, but not limited to, bone marrow (BM), umbilical cord (UC), adipose tissue, and umbilical cord blood (UCB).

The terms “skin rejuvenation” as used herein refers to a process of making the skin, particularly the facial, neck, chest and back skin, look better and more youthful by exerting a beneficial biological effect on the skin.

A “beneficial biological effect” as used herein refers to exhibition of an effect that is associated with skin rejuvenation. Examples of beneficial biological effects include, but are not limited to, upregulation of fibroblast density; increased proliferation of fibroblasts; diminishing of hyperpigmentation; diminishing or elimination of fine lines and wrinkles including both mimetic and superficial wrinkles; smoothening of the epidermis; increased elasticity of the epidermis; increased collagen production; increased hyaluronic acid production; increased elastin production; regeneration of the ECM; etc.

The term “scaffolding” as used herein refers to a framework or structural element that provides support for cells in a specific place. As used herein, the framework of the hyaluronic acid composition in combination with the isotonic sterile saline mist supports the stem cells to hold them within the microperforations in the skin which allows the stem cells to penetrate into the dermis and increase fibroblast density and proliferation in the dermis. The scaffolding itself extends into the dermis.

The term “hyaluronic acid composition” as used herein refers to a composition such as a lotion, cream, serum, or ointment containing hyaluronic acid. Generally, the composition contains 3 mg of HA per cc of normal saline, however a range of between about 1.5 mg and about 5 mg are contemplated.

The term “fluid” or “liquid” as used herein refers to a substance that flows freely but is of constant volume and has a consistency of water or oil. Liquids/fluids have the thinnest consistency as compared to serums, lotions, creams and ointments. Consistency from thinnest to thickest is fluid/liquid->serums->lotions->gels->creams->ointments.

The term “serum” as used herein refers to a watery fluid formulated with a high concentration of active ingredients so that a few drops are enough to cover a large treatment area such as the face.

The term “lotion” as used herein refers to a water-soluble thick liquid preparation capable of being applied to the skin.

The term “gel” as used herein refers to a water-soluble, thick, jelly-like composition capable of being applied to the skin. Gels are less opaque and lighter than creams, generally containing less oil than creams.

The term “cream” as used herein refers to a water-soluble, opaque, thick liquid or semisolid composition capable of being applied to the skin. Creams have a thicker consistency as compared to lotions and serums. Creams contain more oil than a lotion but less than an ointment.

The term “ointment” as used herein refers to a semisolid composition that is thicker than a cream and generally has a higher concentration of oils as compared to a cream. Ointments are not water-soluble due to the amount of oil contained therein.

The term “wrinkle” as used herein refers to a lines, folds, furrows, or creases in the skin. Wrinkles are caused by thinning of the dermal layer of the skin as part of the aging process, damage from environmental factors such as the sun or smoking, and repeated facial movements. “Superficial wrinkle” refers to fine lines limited to superficial dermal creasing. “Mimetic wrinkle” refers to lines (partial thickness) or furrows (full thickness) formed from deep dermal creasing caused by repeated facial movement and expression combined with dermal elastosis. Glabellar lines form from frequent frowning while periorbital lines and nasolabial folds result from smiling. Radial lip and marionette lines are formed from movement of the mimetic muscles during chewing. Mimetic wrinkles do not respond to resurfacing procedures and are usually treated by injectables or muscle resection. Unless otherwise indicated the term “wrinkle” encompasses both superficial and mimetic wrinkles.

The term “hyperpigmentation” as used herein refers to one or more patches of skin that become darker as compared to the normal surrounding skin and is caused by an increase in melanin due to age, hormones, sun damage, skin injuries, etc.

The term “dressing” as used herein refers to an apparatus used to hold the silicone mask in place over the treatment area without disturbing either the mask or the treatment area. In some embodiments, the dressing is rolled gauze which is wrapped around the head of the patient to hold the mask in place on the treatment area. In other embodiments, the dressing is a tubular elastic retainer net bandage that fits around the patient's head.

The term “covering” as used herein refers to a thin medical grade sheet material designed to be placed over the treatment area of the patient so that it is in direct contact with the treatment area. In some embodiments, the covering is manufactured from silicone. The covering must be pH neutral (pH of 7). The terms “covering” and “mask” are used interchangeably herein. In some embodiments the mask is for the face with holes for the eyes, nostrils and mouth being pre-cut while in other embodiments, the holes may be cut after placement on the patient. In some embodiments, the mask is a solid sheet that can be sized for placement on other body parts that are treated such as the neck, back and chest.

The term “post procedure skin healing composition” as used herein refers to a topical lotion, cream or ointment to be applied after treatment for the prevention or relief of dryness and/or protection of the skin. The post-procedure skin healing composition preferably reduces inflammatory tissue swelling, skin redness and blotchiness and prevents degradation of collagen produced by the procedure. Optionally, the post-procedure skin healing cream may also contain ingredients which reduce post-procedure pain. Preferably, the composition contains skin healing components including, but not limited to, aloe vera and/or emollients such as petroleum.

An “emollient” as used herein is defined as a compound used for the prevention or relief of dryness, as well as for the protection of the skin. A wide variety of suitable emollients are known and may be used herein (Sagarin, Cosmetics Science and Technology, 2nd Edition, Vol. 2, pp. 443-465 (1972)). Examples of classes of useful emollients include the following: (1) hydrocarbon oils and waxes such as mineral oil, petrolatum, paraffin, ceresin, ozokerite, microcrystalline wax, polyethylene, and perhydrosqualene; (2) silicone oils, such as dimethyl polysiloxanes, methylphenyl polysiloxanes, water-soluble and alcohol-soluble silicone glycol copolymers; (3) triglyceride esters, such as vegetable and animal fats and oils including castor oil, safflower oil, cottonseed oil, corn oil, olive oil, cod liver oil, almond oil, avocado oil, palm oil, sesame oil, and soybean oil; (4) acetoglyceride esters, such as acetylated monoglycerides; (5) ethoxylated glycerides, such as ethoxylated glyceryl monostearate; (6) alkyl esters of fatty acids having 10 to 20 carbon atoms such as Examples of other useful alkyl esters include hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate, of which methyl, isopropyl, and butyl esters of fatty acids are particularly useful; (7) alkenyl esters of fatty acids having 10 to 20 carbon atoms such as oleyl myristate, oleyl stearate, and oleyl oleate; (8) fatty acids having 10 to 20 carbon atoms such as pelargonic, lauric, myristic, palmitic, stearic, isostearic, hydroxystearic, oleic, linoleic, ricinoleic, arachidic, behenic, and erucic acids; (9) fatty alcohols having 10 to 20 carbon atoms such as lauryl, myristyl, cetyl, hexadecyl, stearyl, isostearyl, hydroxystearyl, oleyl, ricinoleyl, behenyl, erucyl alcohols, and 2-octyl dodecanol; (10) fatty alcohols ethers including ethoxylated fatty alcohols of 10 to 20 carbon atoms such as the lauryl, cetyl, stearyl, isostearyl, oelyl, and cholesterol alcohols having attached thereto from 1 to 50 ethylene oxide groups or 1 to 50 propylene oxide groups; (11) ether-esters such as fatty acid esters of ethoxylated fatty alcohols; (12) lanolin and its derivatives such as lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, ethoxylated sorbitol lanolin, and liquid and semisolid lanolin absorption bases; (13) polyhydric alcohols and polyether derivatives such as propylene glycol, dipropylene glycol, polypropylene glycols 2000 and 4000, polyoxyethylene glycols, polyoxypropylene polyoxyethylene glycols, glycerol, sorbitol, ethoxylated sorbitol, hydroxypropyl sorbitol, polyethylene glycols 200-6000, methoxy polyethylene glycols 350, 550, 750, 2000 and 5000, poly[ethylene oxide]homopolymers (100,000-5,000,000), polyalkylene glycols and derivatives, hexylene glycol (2-methyl-2,4-pentanediol), 1,3-butylene glycol, 1,2,6-hexanetriol, ethohexadiol USP (2-ethyl-1,3-hexanediol), C.sub.15-C.sub.18 vicinal glycol, and polyoxypropylene derivatives of trimethylolpropane; (14) polyhydric alcohol esters including ethylene glycol mono- and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters; (15) wax esters such as beeswax, spermaceti, myristyl myristate, and stearyl stearate; (16) beeswax derivatives, such as polyoxyethylene sorbitol beeswax which are reaction products of beeswax with ethoxylated sorbitol of varying ethylene oxide content, forming a mixture of ether-esters; (17) vegetable waxes including carnauba and candelilla waxes; (18) phospholipids, such as lecithin and derivatives; (19) sterols such as cholesterol and cholesterol fatty acid esters; and (20) amides such as fatty acid amides, ethoxylated fatty acid amides, and solid fatty acid alkanolamides. Particularly useful emollients which provide skin conditioning are glycerol, hexanetriol, butanetriol, lactic acid and its salts, urea, pyrrolidone carboxylic acid and its salts, amino acids, guanidine, diglycerol and triglycerol.

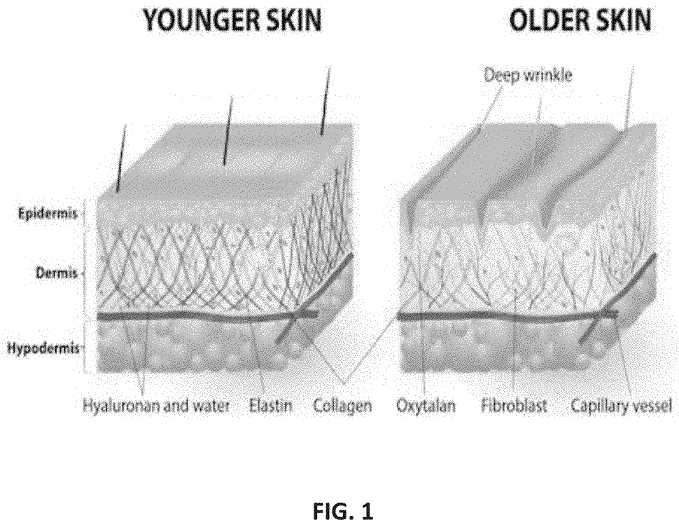

The inventors have developed a method and system capable of reversing the facial aging process by reducing the depth and number of wrinkles, reducing hyperpigmentation, and restoring the elasticity of the skin by increasing the number of fibroblasts in the dermis of the skin. Fibroblasts are cells that constitute the dermal tissue matrix which is comprised of collagen, elastin, and hyaluronic acid. Over time, the number of fibroblasts is reduced as skin ages thus reducing the amount of collagen, elastin and hyaluronic acid in the dermal layer. (FIG. 1). As the density of the fibroblasts increases, the epidermis appears smoother, resulting in a more youthful appearance with skin that is more elastic.

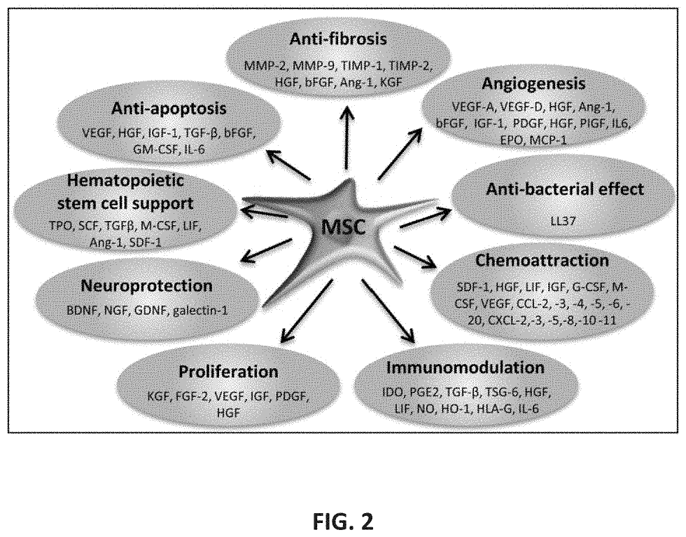

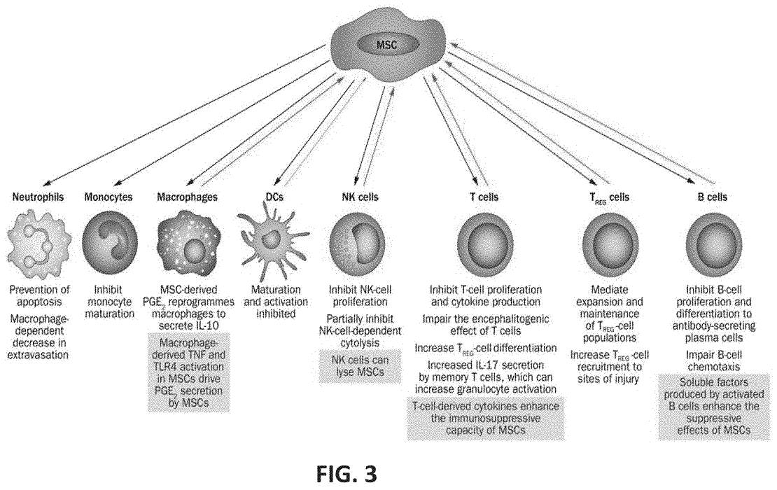

The method engrafts stem cells into the skin's dermis where the stem cells can secrete trophic factors to change the microenvironment of the dermis and increase the number of fibroblasts which, in turn, increases the collagen and elastin density in the dermis thus leading to an improved skin appearance by diminishing the signs of aging such as wrinkles and hyperpigmentation. Examples of trophic factors secreted by the MSC secretome are shown in FIG. 2 while the different types of cells that can be differentiated from MSC are shown in FIG. 3.

By engrafting the stem cells directly into the dermis of the skin, the stem cells remain viable for weeks as opposed to only hours which allows for longer lasting results (years as opposed to months) as compared to current non-surgical techniques to combat the skin's appearance in response to aging. Because the actual number and density of fibroblasts is increased in the dermal layer, more effective results are also obtained as compared to current non-surgical techniques due to the stem being actual living cells capable of rebuilding the ECM and secreting trophic factors.

The method generally includes numbing and cleansing the area followed by application of a hyaluronic acid composition to form a scaffolding for the stem cells. The skin is then microneedled and stem cells are applied to the treated areas. Once the stem cells are deposited within the treated area, a covering, such as a silicon sheet mask, is applied to the area, making complete and uniform contact with the treated skin. A dressing is then applied over the covering and remains on the patient for at least 6 hours to ensure engraftment of the stem cells in the dermis.

### Example 1

The patient is prepped for the procedure by placing a headband along the hairline to hold back the patient's hair from the face followed by a cap over the hair. A topical anesthetic, such as a numbing cream, is applied in a thick, even layer to the treatment area with the cream being massaged into the skin. The topical anesthetic is allowed to penetrate into the skin for between about 50-60 minutes without disturbing the treatment area. It is critical that the patient not disturb the treatment area. Once the topical anesthetic has penetrated the skin, excess cream can be removed by wiping with a water-moistened pad.

The skin of the treatment area is then cleansed with a gentle cleanser, once by hand and the second time with an ultrasonic facial brush to ensure removal of all topical anesthetic from the treatment area and then patted dry with a clean cloth. The entire treatment area is then cleaned thoroughly, first with warm water and second with alcohol wipes and then allowed to air dry.

After cleansing, the entire treatment area is moisturized first with isotonic sterile saline mist. After application of the isotonic sterile saline mist, the treatment area is left to air dry. A hyaluronic acid composition, such as a hyaluronic acid cream, having a physiologic pH of 7 is then applied to a first section of the treatment area, which normally constitutes the forehead, nose, periocular area and upper cheeks. The skin is again moisturized with isotonic sterile saline mist. The combination of the isotonic sterile saline mist and hyaluronic acid composition are critical to maintaining the stem cells viability and should not be substituted. These specific components act as a scaffolding to assist stem cell engraftment in the treatment area.

The skin of the first section of the treatment area is then punctured with microneedles to form a plurality of microperforations. In some embodiments, microneedling is used to form the microperforations in which a device having a plurality of ultra-fine microneedles is used which do not significantly damage the epidermis. Preferably, a microneedling device capable of allowing for varying penetration depths between about 0.5 mm and 2.5 mm is used. Each treatment section is covered with at least 4, but not more than 6, passes of the microneedling device. Specific areas of concern can be spot-treated with additional passes at deeper needle depths of 2.5 mm as tolerated. These microperforations allow for the transdermal delivery of stem cells to the dermal layer of the skin. Microneedling may produce a slight increase in the production of collagen, however when combined with stem cell engraftment in the dermal layer of the skin, at least an additive, if not a synergistic, increase in collagen production occurs.

Once microneedling of the first section of the treatment area is completed, the isotonic sterile saline mist is sprayed again on the first section of the treatment area and the skin is lightly blotted with sterile pads to remove any excess blood. A second layer of the hyaluronic acid composition is applied to the first section of the treatment area and distributed very gently over the first section of the treatment area by gloved fingertips. This second application of isotonic sterile saline and hyaluronic acid composition, in combination with the first application of the isotonic sterile saline and hyaluronic acid, assists in constructing the scaffolding used to support the stem cells within the microperforations which allows the stem cells to remain within the microperforations to engraft within the dermal layer of the skin.

Stem cells are then applied to the first section of the treatment area which has been microneedled, ensuring that the entire first section of the treatment area is covered with the stem cells. If stem cells are topically applied as a fluid, the stem cell fluid may be gently massaged into the skin by gloved fingertips.

Preferably human umbilical cord blood cells (hUCBCs) are administered, however the use of other types of stem cells is contemplated by the invention. hUCBCs may be purchased from a facility or alternatively may be isolated from the umbilical cord following the birth of a child and prepared by known procedures. In some embodiments, human umbilical cord blood derived mesenchymal stem cells (hUCB-MSCs) are used.

Stem cells for the Examples were procured from the Utah Cord Bank (Utah Cord Bank, Inc., Sandy, Utah). Cells were prepared from umbilical cord blood from donated umbilical cords that are procured and collected after full-term, live births. After preparation, cells are cryopreserved until use.

Preferably, at least one million (1×10⁶) stem cells are administered, however dosages between 1×10⁶ and 10×10⁶ may be used for the face area. Maximum surface area per treatment is 10 cm×20 cm.

Once the entire first section of the treatment area is covered with stem cells, a sterile covering, such as a silicone sheet mask, is applied to provide complete and uniform contact with the treated skin. The covering assists in ensuring the stem cells remain in the microperforations and protecting the treated area from environmental damage after the procedure. The covering may have precut areas for the eyes, nostrils and lips or alternatively an opening for the eyes may be cut and a cotton-tipped applicator may be used to position the covering around eyelids allowing for movement of eyelids for blinking.

Once the first section of the treatment area has been treated, the second section of the treatment area comprising the lower cheeks, perioral area and jawline is treated using the same enumerated steps described above. For the second section of the treatment area, with regard to the covering, the covering may have a precut area for the lips and nostrils or alternatively, openings for the lips and nostrils may be cut and a cotton-tipped applicator may be used to position the silicone mask around the lips to allow for opening of the mouth and breathing through nostrils. If other areas are to be treated, such as the neck, back and/or chest area or other areas of the body, the steps above are repeated for each additional treatment area. In embodiments in which the neck, back, chest or other body areas are treated, the covering can be a solid sheet without the need for any openings.

Once the entire treatment area has been treated and the covering applied, a dressing such as rolled gauze or a tubular elastic retainer net bandage is applied over the surface of the covering to hold the covering in place over the treatment area without disturbing the covering or the treatment area.

The average treatment time for the face is between about 30-60 minutes, including bandaging. The dressing and covering should remain on the patient for at least 6 hours to ensure stem cell engraftment in the dermal skin layer.

Upon discharge, the patient is given a post-procedure skin healing composition as well as a hyaluronic acid cream with instructions on post-procedure care. For the first few days, both compositions are applied twice daily with the hyaluronic acid composition applied first. It is important that antioxidants, retinols and other facial treatments are avoided for several months post-procedure. The patient should also avoid excessive UV exposure and wear appropriate sun protection factor (SPF) when going outside.

### Example 2

Wrinkles form from the relaxation of the skin caused by receding papillae and degeneration of collagen and elastin fibers in the dermis and at the dermal-epidermal junction. Wrinkles can be assessed by various methods to determine the efficacy of a treatment. Two such methods are (1) Fitzpatrick Wrinkle Assessment Scale and (2) Lemperle Wrinkle Assessment scale. The Fitzpatrick Wrinkle Assessment scale classifies periorbital and perioral wrinkling and is directed towards generalized wrinkling and elastosis and has been used to determine the results from laser resurfacing. The Lemperle Wrinkle Assessment Scale classifies facial wrinkle depth and has been used in determining results from injectables. The Lemperle scale assesses deeper mimetic wrinkles to differentiate these deeper wrinkles, furrows, and creases from generalized elastosis or folds. (Lemperle, G. et al., A classification of facial wrinkles, Plastic and Reconstructive Surgery, November 2001, 108(6):1735-1750)

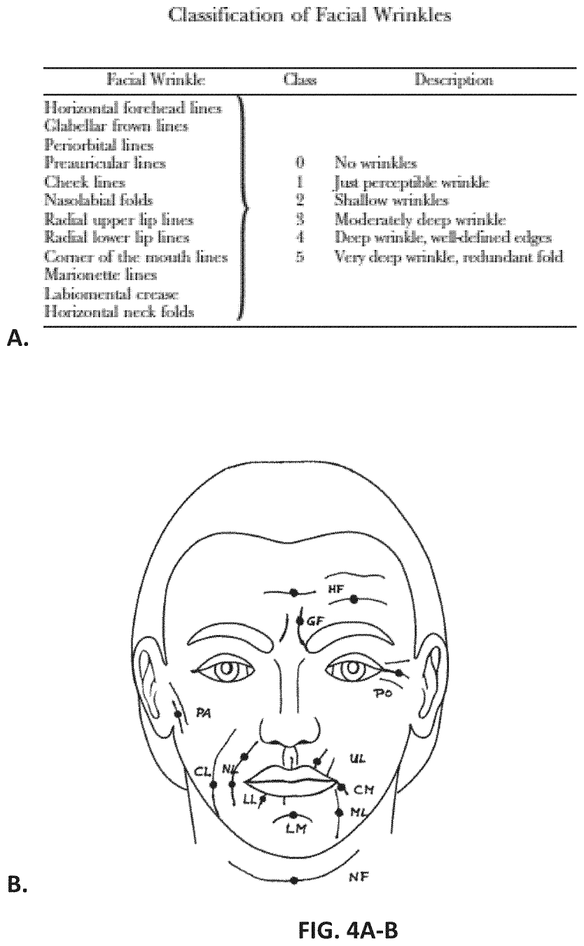

Regardless of the scale used, the assessment is conducted at the same location by the use of anatomic landmarks as shown in FIG. 4B. As shown in FIG. 4B and described in Lemperle 2001, horizontal forehead lines were measured at their intersection with the vertical pupillary line. Glabellar frown lines were measured at the level of the upper border of the eyebrows. Periorbital lines were measured 1.5 cm lateral to the lateral canthus. Preauricular lines were measured at the level of the lower groove of the tragus. Nasolabial folds were measured midway between the alar rim and corner of the mouth (upper nasolabial) and at the level of the corner of the mouth (lower nasolabial). Cheek lines were also measured at the level of the corner of the mouth. The corner of the mouth lines were measured 5 mm below the commissure. Radial lip lines were measured 2 mm above or below the vermilion border. Marionette lines were measured midway between the corner of the mouth and the border of the lower jaw. The labiomental crease and the neck folds were measured in the midline. (Lemperle, G. et al., A classification of facial wrinkles, Plastic and Reconstructive Surgery, November 2001, 108(6):1735-1750)

The treatment of Example 1 was administered to 8 patients (all female, aged 39-75) presenting with facial wrinkles. Prior to beginning the treatment, a wrinkle assessment scale (Lemperle Wrinkle Classification) was used to categorize the wrinkles in the treatment area of each patient. A photograph of the wrinkles in the treatment area is taken prior to initiating treatment. A classification is assigned to each wrinkle in the treatment area based on the categories shown in FIG. 4A. FIG. 4B illustrates the anatomic reference points for the assessment and measurement of wrinkle depth. As used herein, the term “wrinkle” includes superficial wrinkles as well as mimetic wrinkles such as facial furrows, lines and folds.

Efficacy of the treatment was measured at 7 weeks post-treatment and additional images of the treated areas were taken and a new classification score assigned. The inventors were able to quantify improvement in wrinkle appearance for each of the subjects.

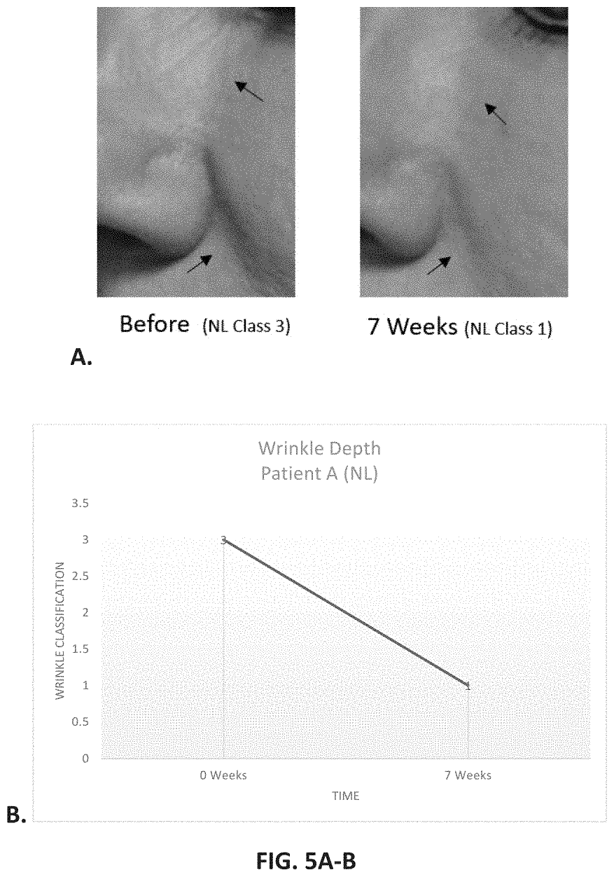

Patient A presented with a score 3 nasolabial folds (NL) as defined by the Wrinkle Assessment Scale depicted in FIG. 4A. Treatment was performed as described in Example 1. At 7 weeks post-treatment, the nasolabial folds (NL) of Patient A had diminished to a score of 1. (FIG. 5A-B)

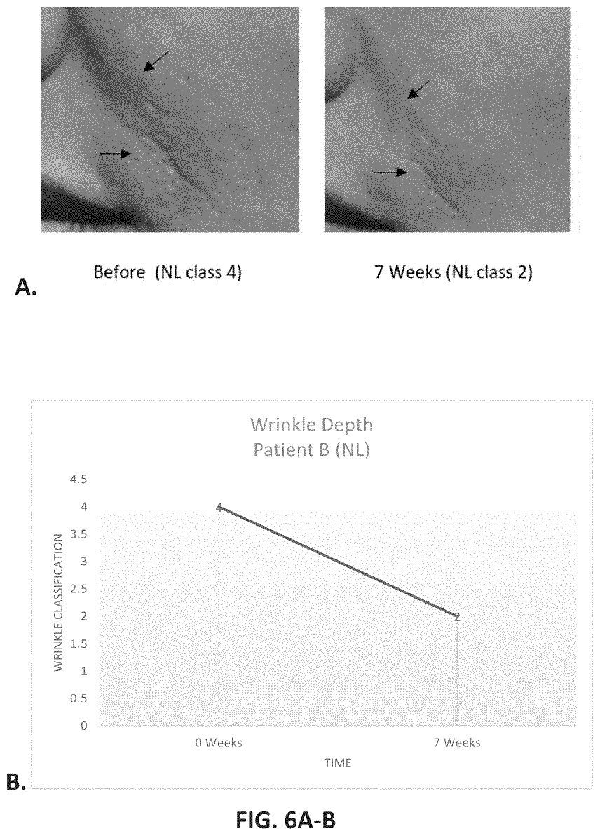

Patient B presented with a score 4 nasolabial folds (NL) as defined by the Wrinkle Assessment Scale depicted in FIG. 4A. Treatment was performed as described in Example 1. At 7 weeks post-treatment, the nasolabial folds (NL) of Patient B had diminished to a score of 2. (FIG. 6 A-B)

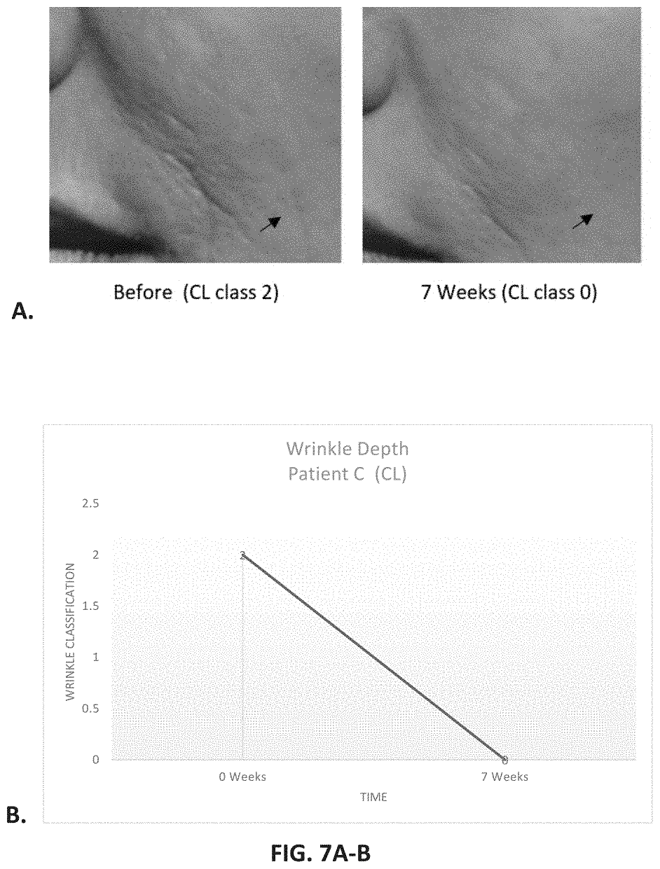

Patient C presented with a score 2 cheek lines (CL) as defined by the Wrinkle Assessment Scale depicted in FIG. 4A. Treatment was performed as described in Example 1. At 7 weeks post-treatment, the cheek lines (CL) of Patient C had diminished to a score of 0. (FIG. 7 A-B)

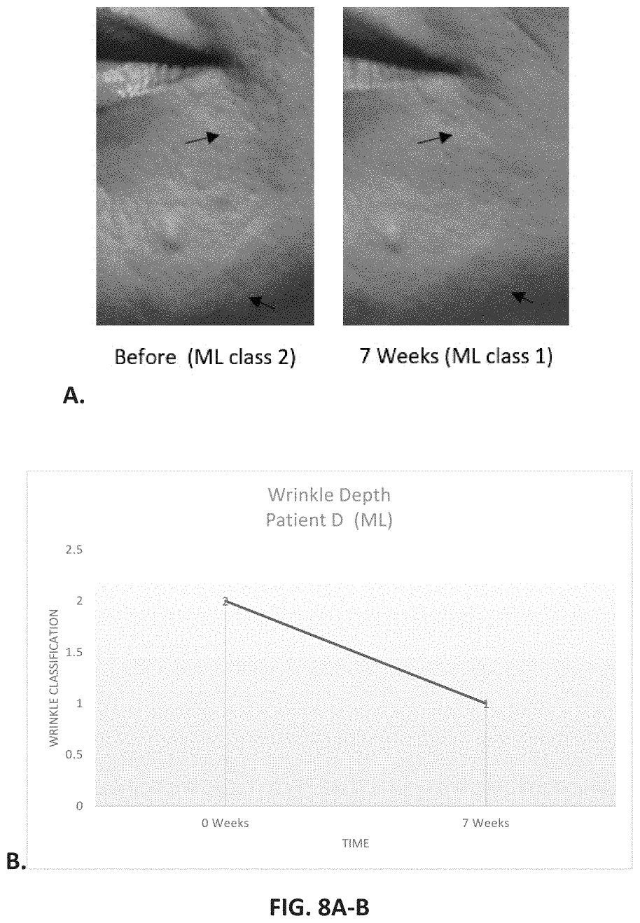

Patient D presented with a score 2 marionette lines (ML) as defined by the Wrinkle Assessment Scale depicted in FIG. 4A. Treatment was performed as described in Example 1. At 7 weeks post-treatment, the marionette lines (ML) of Patient D had diminished to a score of 1. (FIG. 8 A-B)

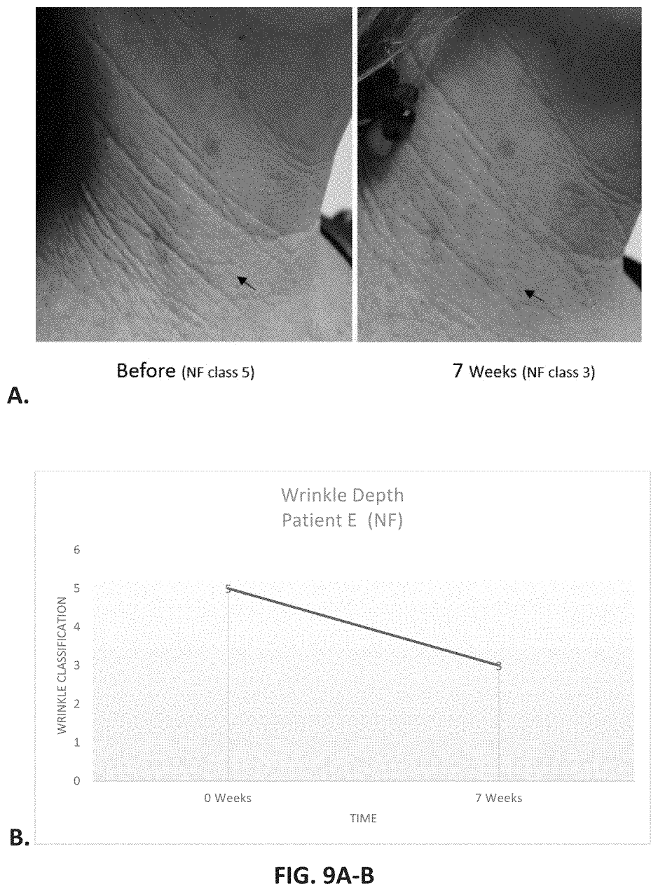

Patient E presented with a score 5 horizontal neck folds (NF) as defined by the Wrinkle Assessment Scale depicted in FIG. 4A. Treatment was performed as described in Example 1. At 7 weeks post-treatment, the horizontal neck folds (NF) of Patient E had diminished to a score of 3. (FIG. 9 A-B)

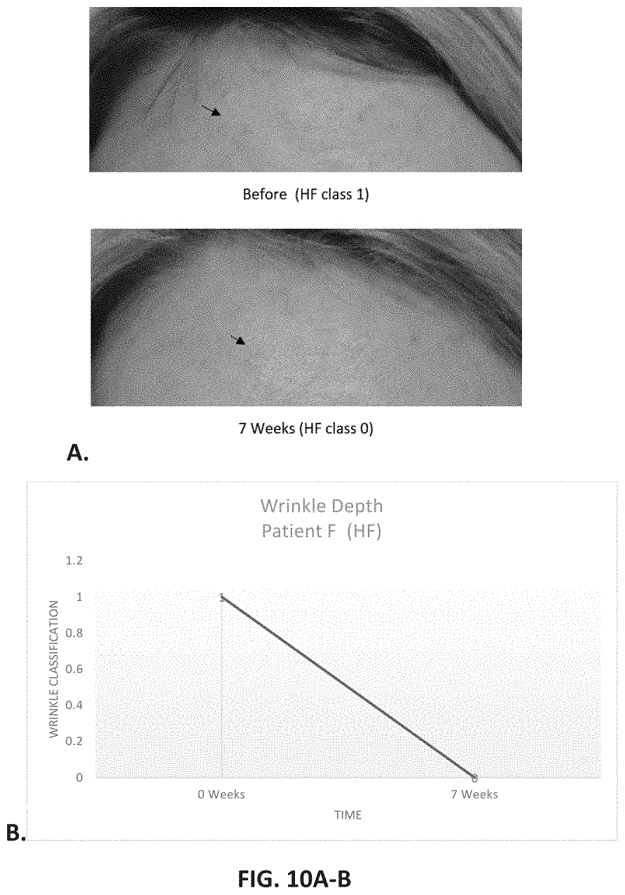

Patient F presented with a score 1 horizontal forehead lines (HF) as defined by the Wrinkle Assessment Scale depicted in FIG. 4A. Treatment was performed as described in Example 1. At 7 weeks post-treatment, the horizontal forehead lines (HF) of Patient F had diminished to a score of 0. (FIG. 10 A-B)

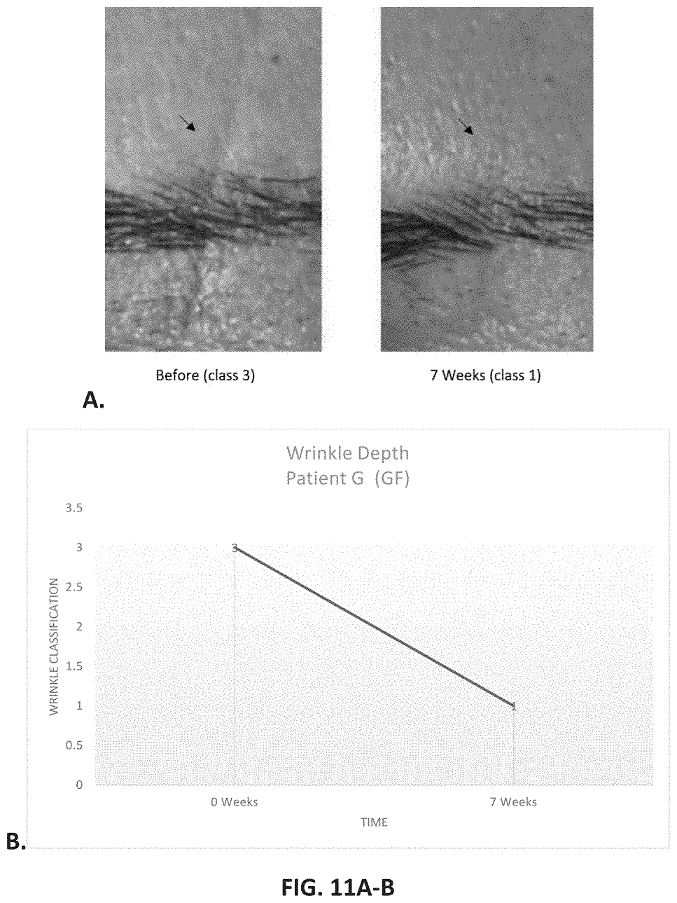

Patient G presented with a score 3 glabellar frown lines (GF) as defined by the Wrinkle Assessment Scale depicted in FIG. 4A. Treatment was performed as described in Example 1. At 7 weeks post-treatment, the glabellar frown lines (GF) of Patient G had diminished to a score of 1. (FIG. 11 A-B)

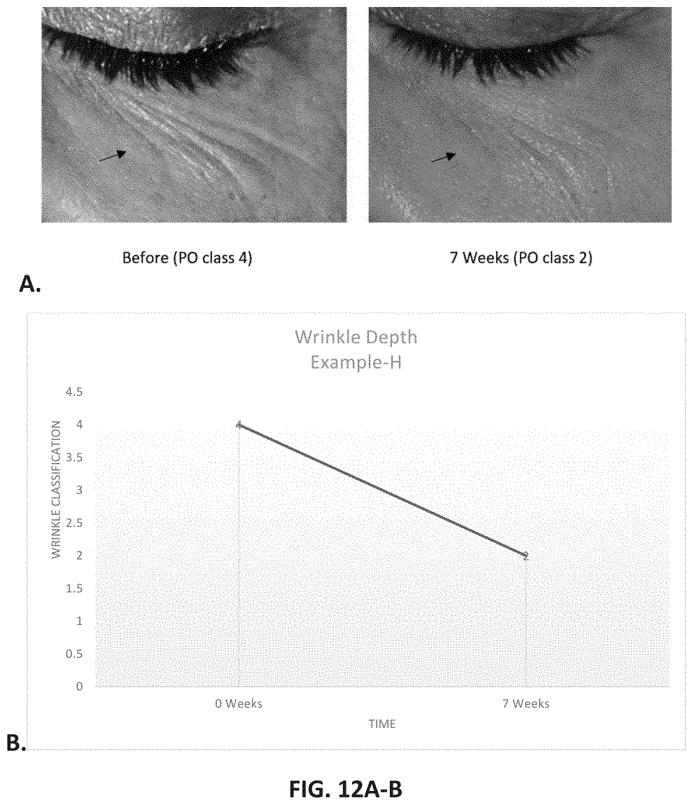

Patient H presented with a score 4 periorbital lines (PO) as defined by the Wrinkle Assessment Scale depicted in FIG. 4A. Treatment was performed as described in Example 1. At 7 weeks post-treatment, the periorbital lines (PO) of Patient A had diminished to a score of 2. (FIG. 12 A-B)

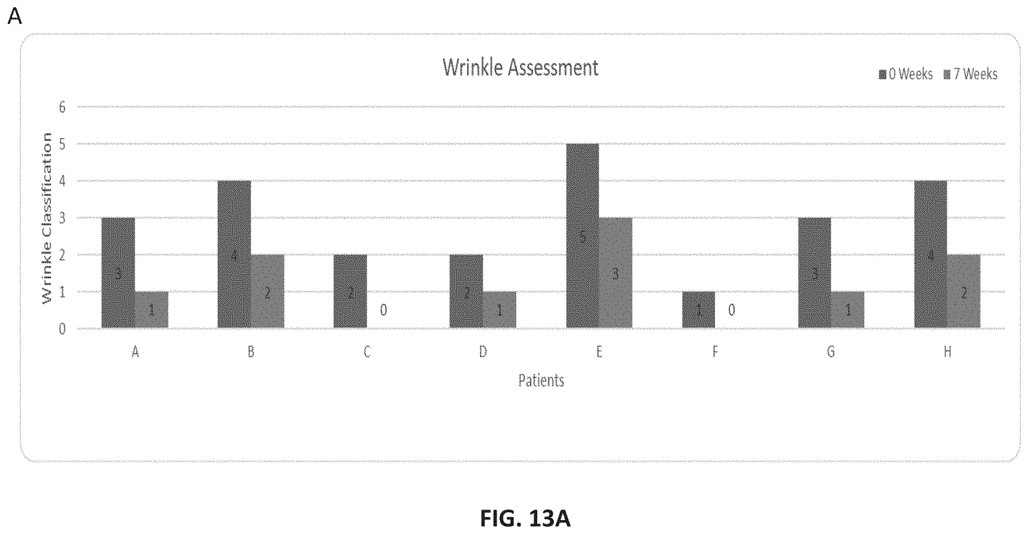

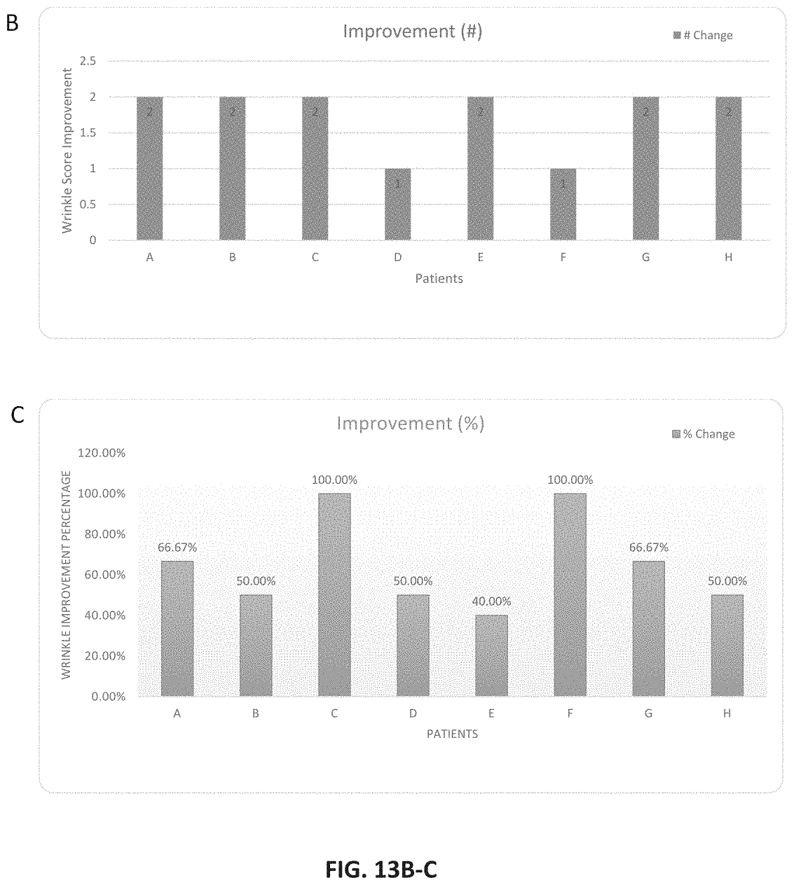

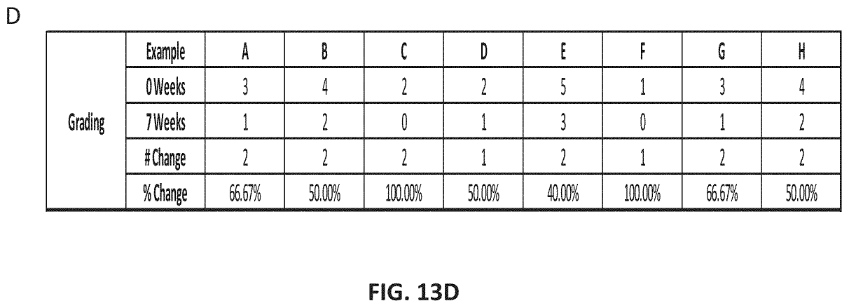

Combined results of Patients A-H are depicted in FIG. 13A-D. FIG. 13(A) is a graph depicting the Lemperle score of Patients A-H before and 7 weeks post-treatment. FIG. 13(B) is a graph depicting the numerical change in the Lemperle score of Patients A-H. As shown in the image, all patients improved at least one classification score, with the majority of patients improving 2 classification scores. FIG. 13(C) is a graph depicting the percentage of improvement in Patients A-H 7 weeks post-treatment. As shown in the image, an improvement of at least 40% is shown 7 weeks post-treatment, with 2 patients (Patients C and F) showing 100% improvement to a score of 0 at 7 weeks post-treatment. FIG. 13(D) table summarizing the results of FIGS. 13(A-C). The inventors have shown that both superficial wrinkles as well as mimetic wrinkles can be effectively treated with the method described herein.

### Example 3

Hyperpigmentation can be measured by the Taylor Hyperpigmentation Scale which is a visual scale for the evaluation of all skin types consisting of a set of 15 uniquely colored skin hue cards spanning the full range of skin hues, each with 10 bands of increasingly darker graduations of skin hues that represent progressive levels of hyperpigmentation. The range of choices in the scale focuses on common skin hues and levels of hyperpigmentation. In use, the subject's skin color is matched to 1 of the 15 laminated skin hue cards in the scale. Once the appropriate skin hue is identified, the hyperpigmentation value (scaled 1-10) that best matches the affected area of hyperpigmentation is selected from the 10 gradations on the corresponding skin hue card. The values for skin hue and hyperpigmentation are then recorded and may be monitored to evaluate the progression of the pigmentation or the effectiveness of therapy. (Taylor, S. C. et al., The Taylor Hyperpigmentation Scale: a new visual assessment tool for the evaluation of skin color and pigmentation, Cutis, 2005, 76:270-274.)

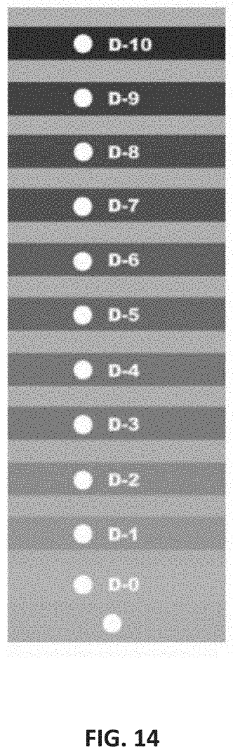

The treatment of Example 1 was administered to 4 patients (all female, aged 39-67) presenting with hyperpigmentation. Prior to beginning treatment, the Taylor Hyperpigmentation Scale was used to categorize the hue of the hyperpigmentation in the treatment area of each patient. (FIG. 14). A photograph of the hyperpigmentation in the treatment area was taken prior to initiating treatment. A classification score is assigned to each hyperpigmentation in the treatment area based on the scoring chart shown in FIG. 14. Efficacy of the treatment was measured at 7 weeks post-treatment and additional images of the treated areas were taken and a new classification score assigned. The inventors were able to quantify improvement in hyperpigmentation appearance for each of the subjects. While the patients are labeled as Patients A-D, please note that the hyperpigmentation patients are not the same as those in the wrinkle assessment of Example 2 above.

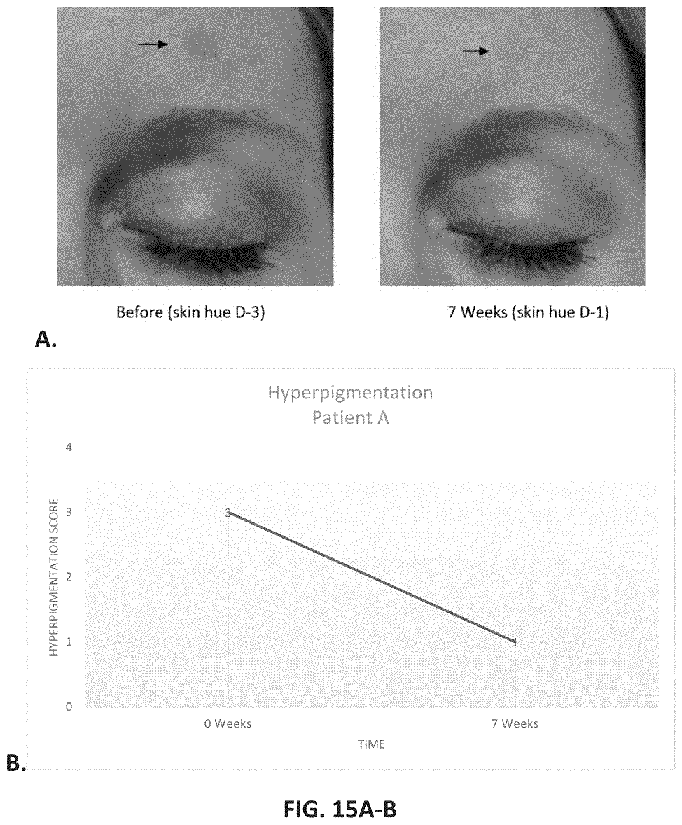

Patient A presented with a skin hue of D-3 as defined by the Taylor Hyperpigmentation Scale depicted in FIG. 14. Treatment was performed as described in Example 1. At 7 weeks post-treatment, the hyperpigmentation of Patient A had diminished to a skin hue of D-1. (FIG. 15 A-B)

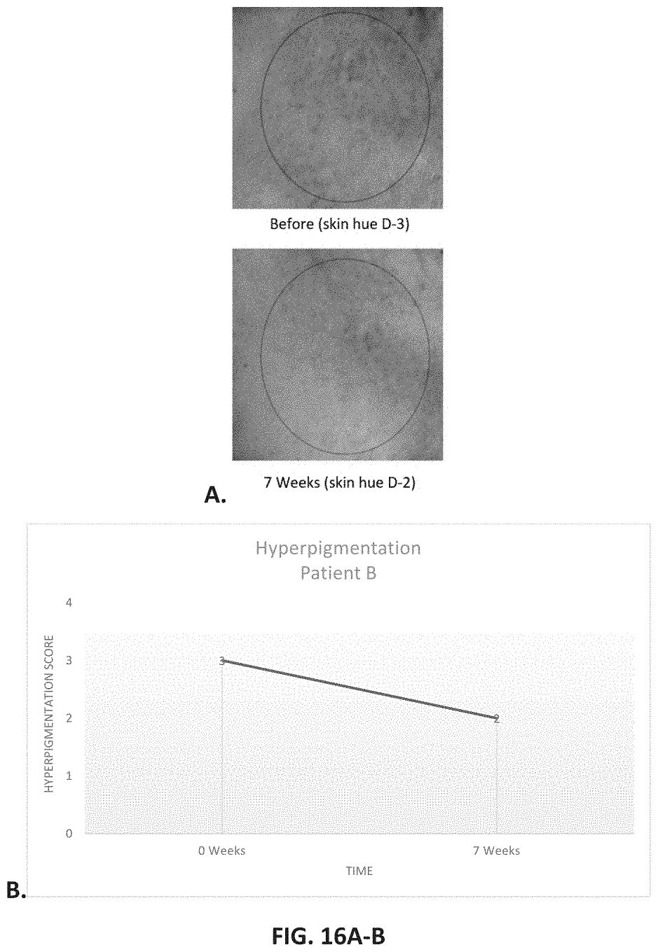

Patient B presented with a skin hue of D-3 as defined by the Taylor Hyperpigmentation Scale depicted in FIG. 14. Treatment was performed as described in Example 1. At 7 weeks post-treatment, the hyperpigmentation of Patient B had diminished to a skin hue of D-2. (FIG. 16 A-B)

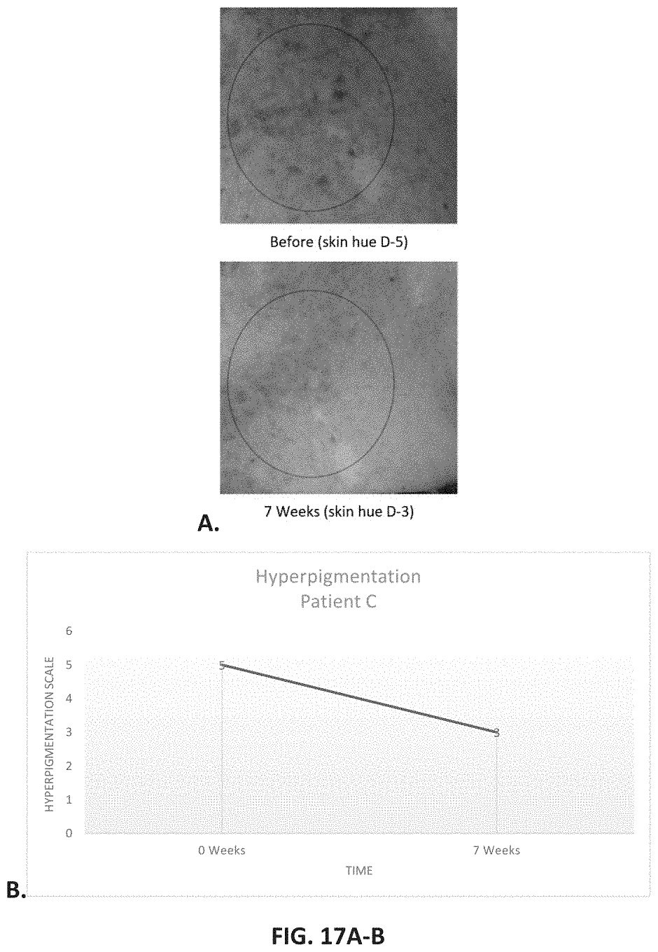

Patient C presented with a skin hue of D-5 as defined by the Taylor Hyperpigmentation Scale depicted in FIG. 14. Treatment was performed as described in Example 1. At 7 weeks post-treatment, the hyperpigmentation of Patient C had diminished to a skin hue of D-3. (FIG. 17 A-B)

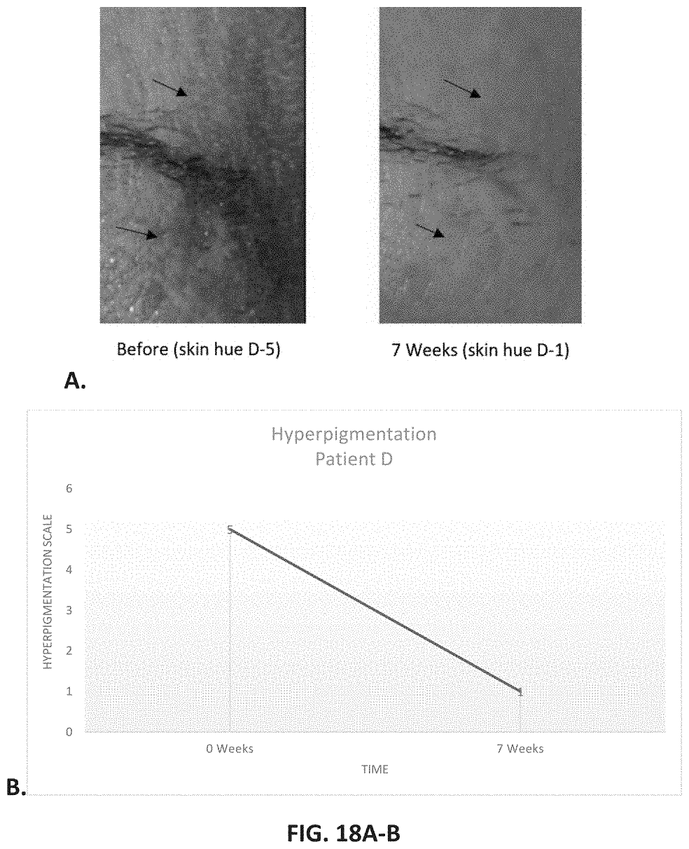

Patient D presented with a skin hue of D-5 as defined by the Taylor Hyperpigmentation Scale depicted in FIG. 14. Treatment was performed as described in Example 1. At 7 weeks post-treatment, the hyperpigmentation of Patient d had diminished to a skin hue of D-1. (FIG. 18 A-B)

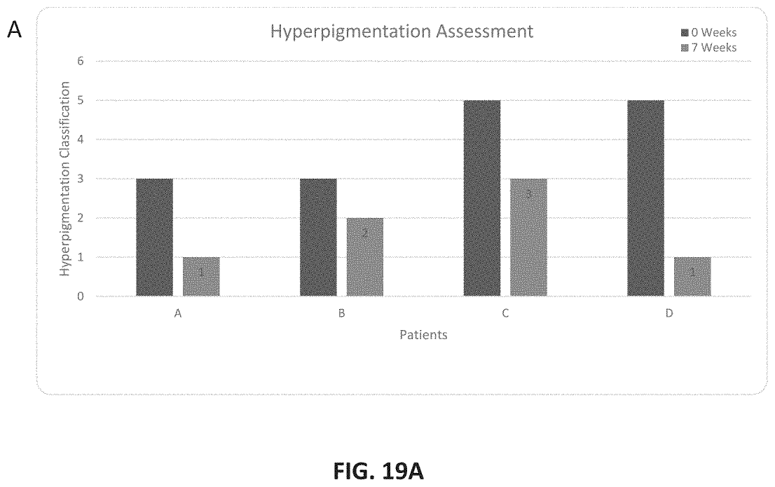

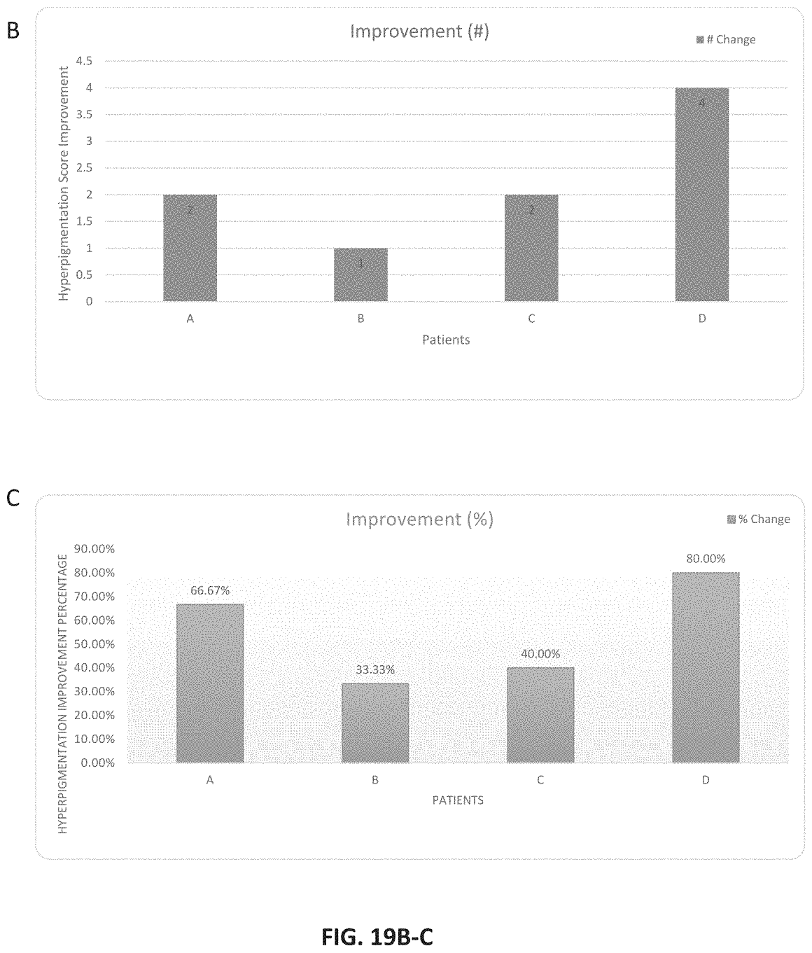

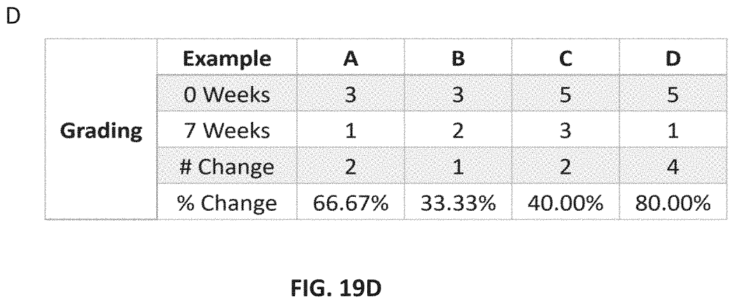

Combined results of Patients A-D are depicted in FIG. 19A-D. FIG. 19(A) is a graph depicting the Taylor score of Patients A-D before and 7 weeks post-treatment. FIG. 19(B) is a graph depicting the numerical change in the Taylor score of Patients A-D. As shown in the image, all patients improved at least one classification score, with the majority of patients improving 2 classification scores. FIG. 19(C) is a graph depicting the percentage of improvement in Patients A-D 7 weeks post-treatment. As shown in the image, an improvement of at least 33% is shown 7 weeks post-treatment, with 1 patient (Patient D) showing 80% improvement at 7 weeks post-treatment. FIG. 19(D) table summarizing the results of FIGS. 19(A-C).

### CONCLUSION

The inventors have developed a novel method and system for reducing and/or eliminating the effects of aging such as wrinkles, loss of elasticity and hyperpigmentation thorough engraftment of stem cells within the dermal layer of the skin which results in an increase in number and density of fibroblasts in the dermal layer. This increase in fibroblast number and density results in increased production of collagen, elastin and hyaluronic acid which improves the texture and pigmentation of the skin. The engraftment of stem cells directly into the dermal layer creates results that can last for years as opposed to the current non-surgical treatments that only last for months.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

The disclosures of all publications cited above are expressly incorporated herein by reference, each in its entirety, to the same extent as if each were incorporated by reference individually.

It is also to be understood that the following claims are intended to cover all of the generic and specific features of the invention herein described, and all statements of the scope of the invention which, as a matter of language, might be said to fall therebetween. Now that the invention has been described,

1. A method of skin rejuvenation in a patient comprising:
topically applying at least one application of isotonic sterile saline to a treatment area of skin of the patient;
topically applying at least one application of a hyaluronic acid composition to the treatment area of the skin of the patient;
microneedling the skin of the treatment area to form a plurality of microperforations wherein the isotonic sterile saline in combination with the hyaluronic acid composition form a scaffolding within the microperforations;
topically administering a therapeutically effective amount of stem cells to the treatment area of the patient wherein the scaffolding supports the stem cells within the microperforations; and
applying a silicone covering having a pH of about 7 on the treatment area for a predetermined amount of time;
wherein the silicone covering holds open the microperforations to allow the stem cells within the microperforations to migrate and engraft in a dermal layer of the skin of the patient.

2. The method of claim 1, further comprising applying a topical anesthetic to the treatment area prior to forming the scaffolding.

3. The method of claim 1, wherein the microneedling is performed with between 4 to 6 passes of a microneedling device over the treatment area.

4. The method of claim 1, wherein the microperforations are between about 0.5 mm to about 2.5 mm in depth.

5. The method of claim 1, wherein the isotonic sterile saline and the hyaluronic acid composition are topically applied both before and after the microneedling.

6. The method of claim 1, wherein the stem cells are umbilical cord derived stem cells or umbilical cord blood derived stem cells.

7. The method of claim 6, wherein the umbilical cord derived stem cells or umbilical cord blood derived stem cells are mesenchymal stem cells.

8. The method of claim 7, wherein at least 1×10⁶ stem cells are administered.

9. The method of claim 1, wherein the covering is a silicone sheeting mask.

10. The method of claim 1, further comprising applying a dressing over the silicone covering to hold the covering in position on the treatment area.

11. The method of claim 10, wherein the silicone covering and dressing remain on the treatment area for at least 6 hours.

12. A method of reducing or eliminating wrinkles in skin of a patient comprising:
topically applying at least one application of an isotonic sterile saline to a treatment area of skin of the patient;
topically applying at least one application of a hyaluronic acid composition to the treatment area of the skin of the patient;
microneedling the skin of the treatment area of the patient to form a plurality of microperforations in the skin of the treatment area of the patient wherein the isotonic sterile saline in combination with the hyaluronic acid composition form a scaffolding within the microperforations;
topically administering a therapeutically effective amount of stem cells to the treatment area of the patient wherein the scaffolding supports the stem cells within the microperforations; and
applying a silicone covering having a pH of about 7 on the treatment area for a predetermined amount of time;
wherein the silicone covering holds open the microperforations to allow the stem cells within the microperforations to migrate and engraft in a dermal layer of the skin of the patient.

13. The method of claim 12, wherein the stem cells are umbilical cord derived stem cells or umbilical cord blood derived stem cells.

14. The method of claim 12, wherein at least 1×10⁶ stem cells are administered.

15. The method of claim 12, wherein the silicone covering is a silicone sheeting mask.

16. The method of claim 12, wherein the silicone covering remains on the treatment area for at least 6 hours.

17. A method of reducing or eliminating hyperpigmentation in skin of a patient comprising:
topically applying at least one application of an isotonic sterile saline to a treatment area of skin of the patient;
topically applying at least one application of a hyaluronic acid composition to the treatment area of the skin of the patient;
microneedling the skin of the treatment area of the patient to form a plurality of microperforations in the skin of the treatment area of the patient wherein the isotonic sterile saline in combination with the hyaluronic acid composition form a scaffolding within the microperforations;
topically administering a therapeutically effective amount of stem cells to the treatment area of the patient wherein the scaffolding supports the stem cells within the microperforations; and
applying a silicone covering having a pH of about 7 on the treatment area for a predetermined amount of time;
wherein the silicone covering holds open the microperforations to allow the stem cells within the microperforations to migrate and engraft in a dermal layer of the skin of the patient.

18. The method of claim 17, wherein the stem cells are umbilical cord derived stem cells or umbilical cord blood derived stem cells.

19. The method of claim 17, wherein at least 1×10⁶ stem cells are administered.

20. The method of claim 17, wherein the silicone covering is a silicone sheeting mask that remains on the treatment area for at least 6 hours.
